(19) United States
(12) Patent Application Publication
(10) Pub. No.: US 2004/0153034 A1
(43) Pub. Date: Aug. 5, 2004

(54) Safety fluid injecting/sampling apparatus

(75) Inventor: Tzu-Sheng Fan, Miao Li Hsien (TW)

Correspondence Address: BACON & THOMAS, PLLC, 625 SLATERS LANE, FOURTH FLOOR, ALEXANDRIA, VA, 22314

(73) Assignee: TAIJECT MEDICAL DEVICE CO., LTD., HSIN CHU HSIEN (TW)

(21) Appl. No.: 10/742,819

(22) Filed: Dec. 23, 2003

(30) Foreign Application Priority Data
Dec. 31, 2002 (CN) 02294498.2
Dec. 31, 2002 (CN) 02294490.7

(51) Int. Cl. A61M 5/32

(52) U.S. Cl. 604/197; 604/110

(57) ABSTRACT

A safety fluid injecting/sampling apparatus connectable with a needle cannula, the apparatus includes an inner barrel having a receiving hole, an elastically bendable retaining strip with an inwardly protruded engagement tooth disposed at one end, and a retaining hole disposed at the other end, an outer barrel having an axially extended receiving hole adapted to receive the inner barrel, a finger flange extended around the periphery, and an elastically deformable retaining element having an engagement block engageable into the retaining hole of the inner barrel; a needle holder axially slidably inserted into the receiving hole of the inner barrel, the needle holder having a locating groove extended around the periphery and adapted to receive the engagement tooth of the inner barrel for positioning, and an insertion member inserted into the receiving hole of the inner barrel for operation by hand to move the outer barrel backwards relative to the inner barrel.

### BACKGROUND OF THE INVENTION

1. Field of the Invention

The present invention relates generally to safety hypodermic syringes and, more particularly, to a safety fluid injecting/sampling apparatus, which enables the needle holder with the needle cannula to be conveniently pulled back and received inside the apparatus after the service of the apparatus.

2. Description of the Related Art

In order to prevent contamination, a hypodermic syringe or blood sampler has the needle cannula made retractable or to be pulled back after the service of the apparatus. However, conventional safety hypodermic syringes or blood samplers are commonly complicated, not easy to fabrication. Further, there are simple designs of safety hypodermic syringes and blood samplers suitable for mass production. However, these simple designs of safety hypodermic syringes and blood samplers are not practical in use, unable to attract medical persons to use.

Therefore, it is desirable to provide a safety fluid injecting/sampling apparatus that eliminates the aforesaid drawbacks.

### SUMMARY OF THE INVENTION

The present invention has been accomplished under the circumstances in view. It is the main object of the present invention to provide a safety fluid injecting/sampling apparatus, which enables the needle holder with the needle cannula to be pulled back and received on the inside after the service of the apparatus. It is another object of the present invention to provide a safety fluid injecting/sampling apparatus, which can conveniently be operated to control positioning/displacement of the needle holder with the needle cannula. It is still another object of the present invention to provide a safety fluid injecting/sampling apparatus, which has a simple structure for normal functioning.

To achieve these and other objects of the present invention, the safety fluid injecting/sampling apparatus comprises an inner barrel, the inner barrel comprising a cylindrical body defining a longitudinally extended receiving hole, an elastically bendable retaining strip extended from a front end of the cylindrical body of the inner barrel and terminating in an inwardly protruded engagement tooth, a retaining hole formed in the periphery of a rear end of the cylindrical body of the inner barrel remote from the retaining strip; an outer barrel, the outer barrel comprising a cylindrical body defining an axially extended receiving hole adapted to receive the cylindrical body of said inner barrel, a finger flange extended around the periphery of the cylindrical body of the outer barrel, and a retaining element elastically deformably formed on the cylindrical body of the outer barrel, the retaining element having an engagement block engageable into the retaining hole of the inner barrel; a needle holder, the needle holder comprising a holder body axially slidably inserted into the receiving hole of the inner barrel, and a locating groove extended around the periphery of the holder body and adapted to receive the engagement tooth of the inner barrel; and an insertion member inserted into the receiving hole of the inner barrel for operation by hand to move the outer barrel backwards relative to the inner barrel. Preferably, the insertion member is a plunger so that the safety fluid injecting/sampling apparatus can function as a hypodermic syringe. The insertion member can be a blood collection tube so that the safety fluid injecting/sampling apparatus can function as a blood sampler.

### BRIEF DESCRIPTION OF THE DRAWINGS

FIG. 1 is an exploded view of a safety fluid injecting/sampling apparatus according to one embodiment of the present invention.

FIG. 2 is a sectional assembly view of the embodiment shown in FIG. 1, showing the status of the safety fluid injecting/sampling apparatus before injection.

FIG. 3 is similar to FIG. 2 but showing the engagement status between the plunger and the needle holder.

FIG. 4 is similar to FIG. 3 but showing the backward pulling status of the outer barrel.

FIG. 5 is similar to FIG. 4 but showing the outer barrel pulled to the back side.

FIG. 6 is similar to FIG. 5 but showing the needle holder disengaged from the constraint of the inner barrel.

FIG. 7 is similar to FIG. 6 but showing the needle holder pulled backwards and received inside of the inner barrel and assumed the breaking status of the plunger.

FIG. 8 illustrates a different arrangement of the needle holder with the needle cannula according to the present invention.

FIG. 9 is a sectional assembly view of an alternate form of the present invention, showing the safety fluid injecting/sampling apparatus made in the form of a blood sampler.

FIG. 10 is an end view of the inner barrel of the blood sampler shown in FIG. 9.

FIG. 11 is an exploded view of another alternate form of the present invention, showing the safety fluid injecting/sampling apparatus made in the form of a hypodermic syringe.

FIG. 12 is a side view in section of the hypodermic syringe shown in FIG. 11.

FIG. 13 shows the needle holder of the hypodermic syringe of FIG. 12 pulled back and received inside the inner barrel.

FIG. 14 is an exploded view of still another alternate form of the present invention, showing the safety fluid injecting/sampling apparatus made in the form of a blood sampler.

FIG. 15 is a side view in section of the blood sampler shown in FIG. 14.

FIG. 16 illustrates the needle holder of the blood sampler of FIG. 15 set in the received status after service.

### DETAILED DESCRIPTION OF THE INVENTION

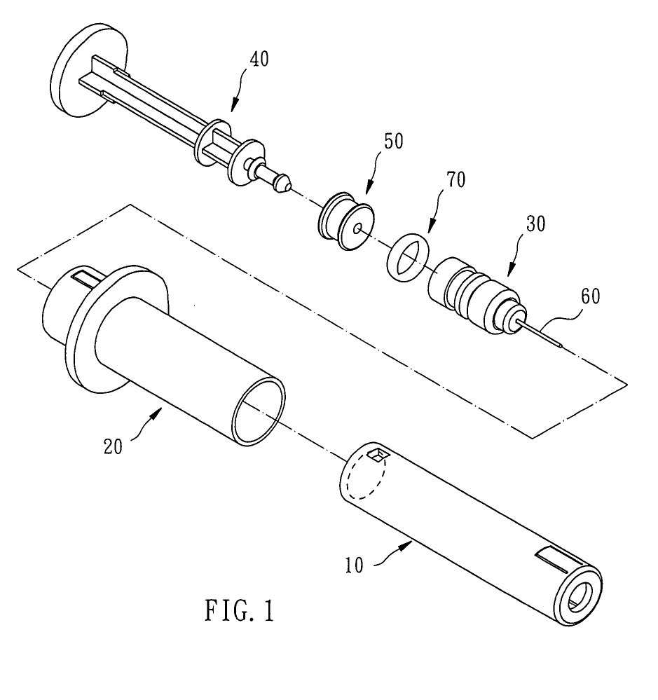

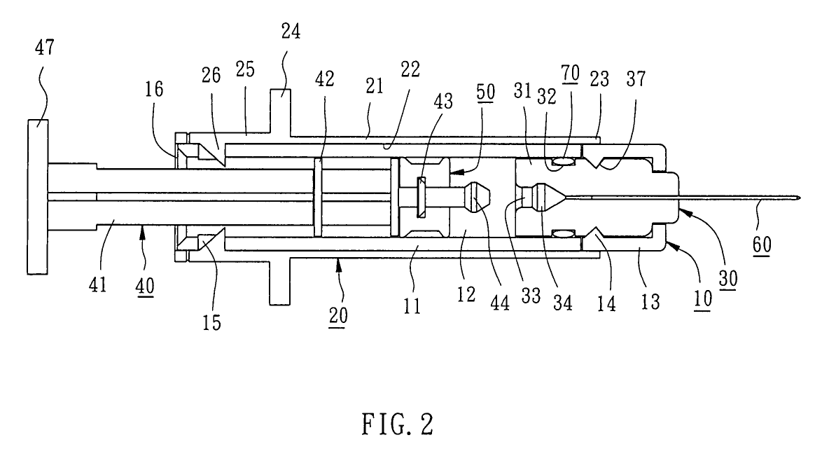

Referring to FIGS. 1 and 2, a safety fluid injecting/sampling apparatus according to the first embodiment of the present invention is a hypodermic syringe mounted with a needle cannula 60. The hypodermic syringe comprises an inner barrel 10, an outer barrel 20, a needle holder 30, a plunger 40, and a stopper 50.

The inner barrel 10 comprises a cylindrical body 11 defining a longitudinally extended receiving hole 12, two elastically bendable retaining strips 13 bilaterally extended from one end, namely, the front end of the cylindrical body 11 in reversed to the extending direction of the needle cannula 60 and terminating in a respectively inwardly protruded engagement tooth 14, two retaining holes 15 bilaterally formed in the periphery of the other end, namely, the rear end of the cylindrical body 11 remote from the retaining strips 13, and an end stop portion 16 formed in the rear end of the cylindrical body 11.

The outer barrel 20 comprises a cylindrical body 21 defining an axially extended receiving hole 22 adapted to receive the cylindrical body 11 of the inner barrel 10, a constraint portion 23 formed in the front end of the cylindrical body 21 and adapted to constrain the retaining strips 13 of the inner barrel 10, a finger flange 24 extended around the periphery of the cylindrical body 21, and two retaining elements 25 elastically deformably formed integral with the cylindrical body 21. The retaining elements 25 each have an engagement block 26 insertable through the locating holes 15 into the inside of the receiving hole 12 of the cylindrical body 11 of the inner barrel 10.

The needle holder 30 comprises a holder body 31 axially slidably inserted into the receiving hole 12 of the inner barrel 10, an annular groove 32 extended around the periphery of the holder body 31 and mounted with an O-ring 70, which is disposed in close contact with the inside wall of the inner barrel 10, a plug hole 33 axially extended from the center of rear end of the holder body 31, a female engagement portion 34 formed in the plug hole 33, and a locating groove 37 extended around the periphery of the holder body 31 and adapted to receive the retaining strips 13 of the inner barrel 10. The front end of the holder body 31 of the needle holder 30 is provided for the mounting of the needle cannula 60 in any of a variety of conventional mounting methods.

The plunger 40 comprises an elongated plunger body 41, a stop flange 42 extended around the periphery of the plunger body 41, a stopper retainer 43 forwardly extended from the front end of the plunger body 41, an male engagement portion 44 forwardly extended from the front end of the stopper retainer 43 and adapted to engage the female engagement portion 34 of the needle holder 30, and a thumb rest 47 formed on the rear end of the plunger body 41 for the rest of the thumb.

The stopper 50 is fastened to the stopper retainer 43 and supported on the front end of the plunger body 41 and peripherally disposed in close contact with the inside wall (the peripheral wall of the receiving hole 12) of the inner barrel 10.

The operation of the present invention is outlined hereinafter. FIG. 2 is an assembly view of the safety fluid injecting/sampling apparatus before use. The status shown in FIG. 2 is similar to the status of use of the safety fluid injecting/sampling apparatus to draw in medicine, or the status of use of the safety fluid injecting/sampling apparatus to inject medicine. Further, when pulling the plunger 40 backwards, medicine is drawn into the inner barrel 10 through the needle cannula 60. At this time, the needle holder 30 is stopped in position inside the front end of the inner barrel 10, the constraint portion 23 of the outer barrel 20 holds down the retaining strips 13 of the inner barrel 10 in engagement with the locating groove 37 of the needle holder 30, and therefore the needle holder 30 is firmed secured in position against vibration, ensuring safety use of the safety fluid injecting/sampling apparatus.

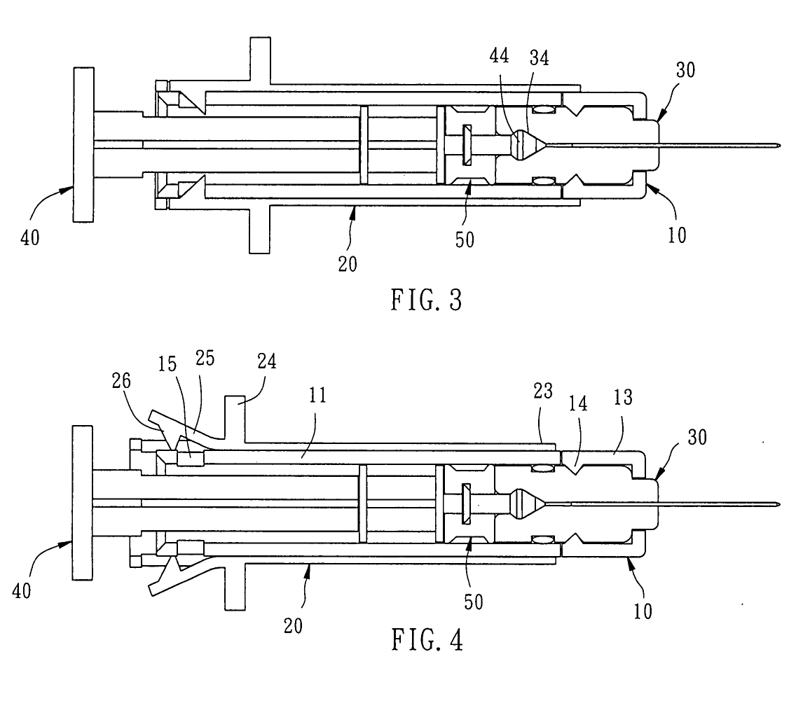

After injection as shown in FIG. 3, the male engagement portion 44 of the plunger 40 is forced into engagement with the female engagement portion 34 of the needle holder 30. At this time, the needle holder 30 is movable with the plunger 40.

When wishing to pull the outer barrel 20 backwards from the inner barrel 10 as shown in FIG. 4, hold the thumb rest 47 and then pull the finger flange 24 to move the outer barrel 20 backwards relative to the inner barrel 10, causing the retaining elements 25 to curve outwards.

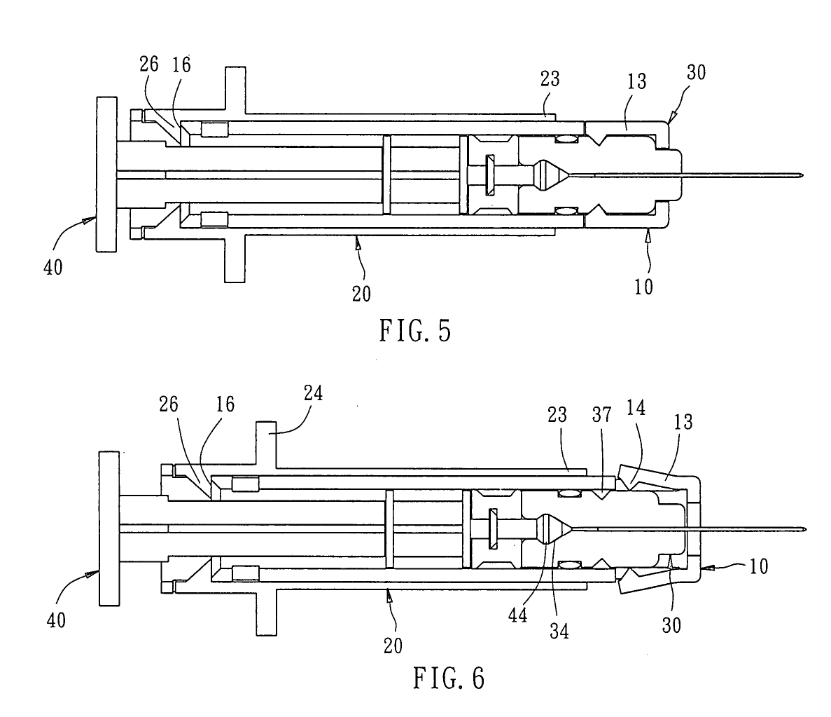

FIG. 5 shows the outer barrel 20 moved to the rear limit position where the constraint portion 23 of the outer barrel 20 is disengaged from the retaining strips 13, and the engagement blocks 26 of the retaining elements 25 are respectively stopped at the end stop portion 16 of the inner barrel 10.

FIG. 6 shows the operation of pulling back the needle holder 30. When pushing the outer barrel 20 forwards after the plunger 40 has been pulled back, the locating groove 37 of the needle holder 30 is disengaged from the engagement teeth 14 of the retaining strips 13 of the inner barrel 10 and the retaining strips 13 are respectively curved outwards, and therefore the needle holder 30 can be moved backwards with the plunger 40.

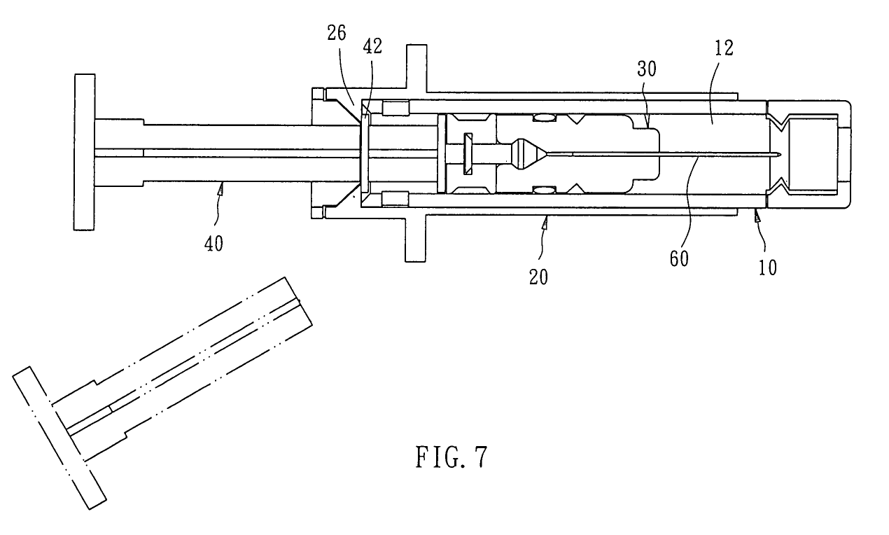

FIG. 7 shows the plunger 40 pulled the needle holder 30 backwards to the inside of the inner barrel 10, the stop flange 42 stopped at the engagement blocks 26 of the retaining elements 25 of the outer barrel 20. At this time, the user can bend the plunger 40 to break the elongated plunger body 41 at the stop flange 42, keeping the front part of the plunger 40, the needle holder 30 and the needle cannula 60 remained inside the receiving hole 12 of the inner barrel 10.

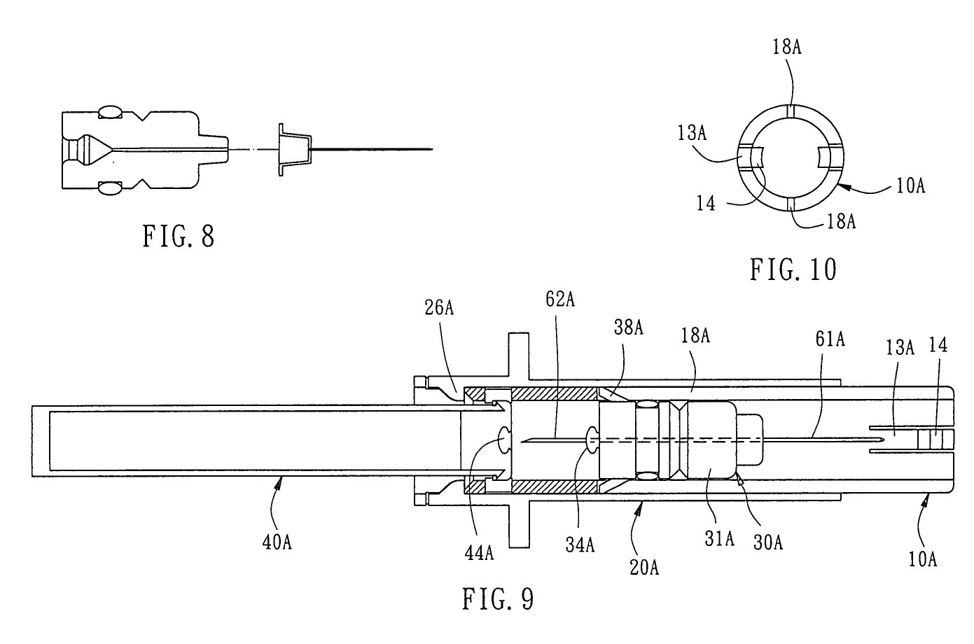

FIG. 8 shows the needle holder adapted to hold a standard detachable needle 10 cannula.

FIGS. 9 and 10 show an alternate form of the safety fluid injecting/sampling apparatus according to the present invention. According to this embodiment, the safety fluid injecting/sampling apparatus is a blood sampler comprising an inner barrel 10A, an outer barrel 20A, a needle holder 30A, and a blood collection tube 40A.

The retaining strips 13A of the inner barrel 1OA extend forwards (reversed to the backward extending design of the retaining strips of the inner barrel of the aforesaid first embodiment of the present invention, each having an inwardly protruded engagement tooth 14. The inner barrel 10A further comprises two locating grooves 18A forming a retaining portion.

The outer barrel 20A has an engagement block 26A, which is not protruded into the inside of the receiving hole of the inner barrel 10A, and the blood collection tube 40A can be inserted and pulled out to the receiving hole of the inner barrel 10A smoothly.

The needle holder 30A comprises a holder body 31A, and two positioning portions 38A respectively movably coupled to the locating grooves 16A. The positioning portions 38A can be stopped at one end of each of the locating grooves 18A. The needle cannula has a first pointed tip 61A for insertion into the patient's blood vessel, and a second pointed tip 61B for insertion into the blood collection tube 40A.

The blood collection tube 40A has a female engagement portion 44A disposed in the front end and adapted to receive a male engagement portion 34A at the rear end of the needle holder 30A.

The operation of this blood sampler is substantially similar to the aforesaid first embodiment of the present invention, and therefore no further detailed description in this regard is needed.

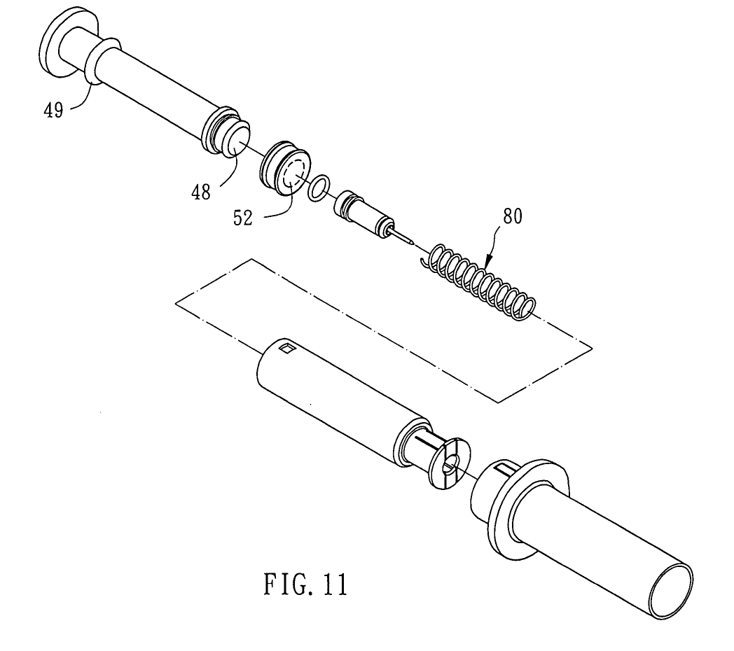

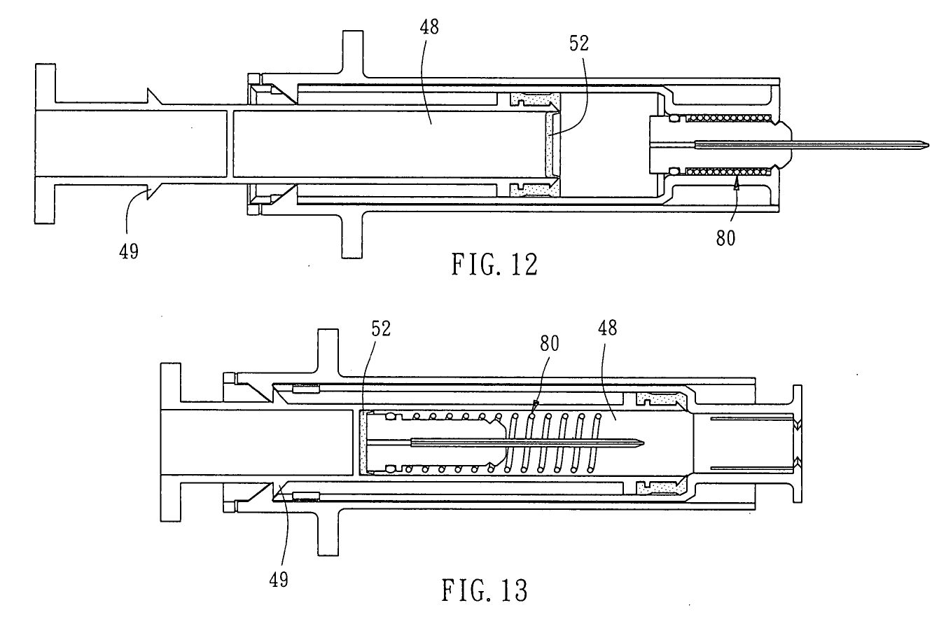

FIGS. 11-13 show another alternate form of the safety fluid injecting/sampling apparatus. According to this embodiment, the safety fluid injecting/sampling apparatus is a safety hypodermic syringe with a retractable needle cannula. This embodiment has different features as outlined hereinafter.

The hypodermic syringe further comprises a spring member 80 mounted in between the inner barrel and the needle holder; the plunger has a receiving hole 48 axially extended to the front end; the stopper has a breaking portion 52 aimed at the receiving hole 48 of the plunger. The breaking portion 52 can be broken by the needle cannula and forced backwards into the inside of the receiving hole 48 of the plunger by the spring member 80. The plunger further comprises a stop flange 49 extended around the periphery. During forward stroke of the plunger, the stop flange 49 will be stopped by a part of the inner barrel. During backward stroke after operation, the stop flange 49 will be stopped at the engagement blocks of the outer barrel.

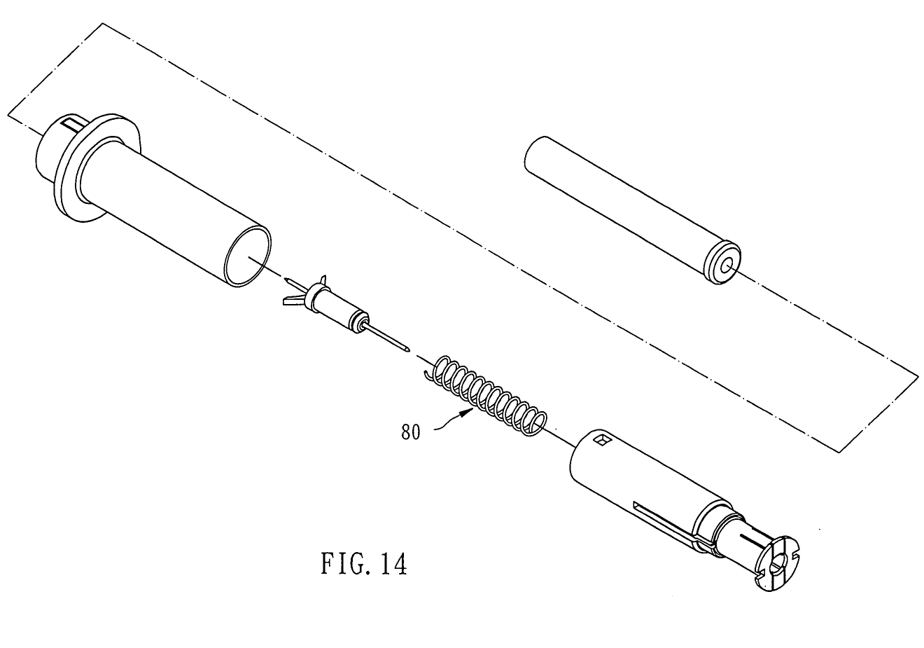

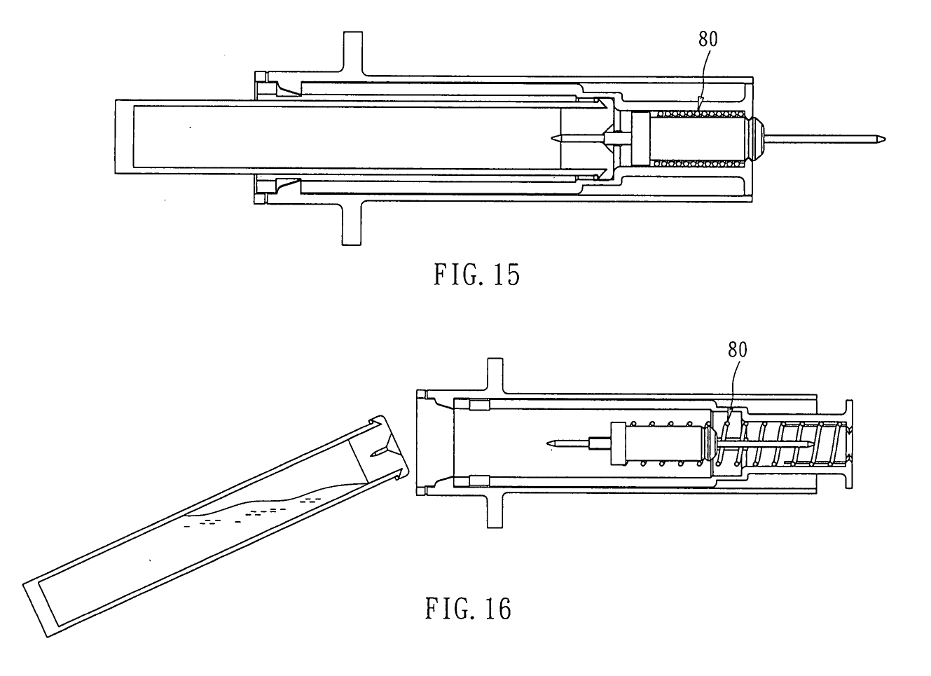

FIGS. 14-16 show the safety fluid injecting/sampling apparatus made in the form of a blood sampler.

As indicated above, the invention has the following characteristics:

1. By means of moving the outer barrel axially, the positioning or displacement of the needle holder is conveniently controlled.

2. The needle holder engagement control is a simple push-and-pull operation, which is easy to achieve.

3. The combination of the outer barrel and the inner barrel is a simple arrangement for convenient operation.

What is claimed is:

1. A safety fluid injecting/sampling apparatus comprising:
an inner barrel, said inner barrel comprising a cylindrical body defining a longitudinally extended receiving hole, an elastically bendable retaining strip extended from a front end of the cylindrical body of said inner barrel and terminating in an inwardly protruded engagement tooth, a retaining hole formed in the periphery of a rear end of the cylindrical body of said inner barrel remote from said retaining strip;
an outer barrel, said outer barrel comprising a cylindrical body defining an axially extended receiving hole adapted to receive the cylindrical body of said inner barrel, a finger flange extended around the periphery of the cylindrical body of said outer barrel, and a retaining element elastically deformably formed on the cylindrical body of said outer barrel, said retaining element having an engagement block engageable into the retaining hole of said inner barrel;
a needle holder, said needle holder comprising a holder body axially slidably inserted into the receiving hole of said inner barrel, and a locating groove extended around the periphery of said holder body and adapted to receive the engagement tooth of said inner barrel; and
an insertion member inserted into the receiving hole of said inner barrel for operation by hand to move said outer barrel backwards relative to said inner barrel.

2. The safety fluid injecting/sampling apparatus as claimed in claim 1, wherein said insertion member is a plunger, and said needle holder holds a needle cannula for enabling the safety fluid injecting/sampling apparatus to function as a hypodermic syringe.

3. The safety fluid injecting/sampling apparatus as claimed in claim 2, wherein said plunger has a receiving hole axially extended to a front end thereof and adapted to receive said needle holder.

4. The safety fluid injecting/sampling apparatus as claimed in claim 1, wherein said insertion member is a blood collection tube, and said needle holder is mounted with a blood sampling needle, for enabling the safety fluid injecting/sampling apparatus to function as a blood sampler.

5. The safety fluid injecting/sampling apparatus as claimed in claim 1, wherein said insertion member is adapted to pull said needle holder backwards to the inside of said inner barrel.

6. The safety fluid injecting/sampling apparatus as claimed in claim 5, wherein said insertion member comprises an engagement portion, and said needle holder comprises an engagement portion adapted to receive the engagement portion for enabling said needle holder to be pulled backwards with said insertion member into the inside of said inner barrel.

7. The safety fluid injecting/sampling apparatus as claimed in claim 1, wherein said needle holder is automatically retractable.

8. The safety fluid injecting/sampling apparatus as claimed in claim 1, wherein said inner barrel comprises a locating groove, said needle holder comprises a positioning portion adapted to engage the locating groove of said inner barrel.

9. The safety fluid injecting/sampling apparatus as claimed in claim 1, wherein the engagement block of said outer barrel projects into the receiving hole of the cylindrical body of said inner barrel at a distance.

10. The safety fluid injecting/sampling apparatus as claimed in claim 9, wherein said insertion member comprises a stop flange adapted to stop said insertion member inside said outer barrel.
